(19) United States
(12) Patent Application Publication
(10) Pub. No.: US 2024/0293270 A1
(43) Pub. Date: Sep. 5, 2024

(54) ABSORBENT ARTICLE

(71) Applicant: The Procter & Gamble Company, Cincinnati, OH (US)

(72) Inventors: Fengchun YE, Beijing (CN); Haoran ZHAO, Beijing (CN)

(21) Appl. No.: 18/663,455

(22) Filed: May 14, 2024

Related U.S. Application Data
(63) Continuation of application No. PCT/CN2021/141087, filed on Dec. 24, 2021.

(51) Int. Cl. A61F 13/84; A61F 13/49

(52) U.S. Cl. CPC A61F 13/84; A61F 13/49011; A61F 2013/8497

(57) ABSTRACT

The present disclosure relates to an absorbent article comprising left and right side panels in a front belt region or a back belt region comprising a first visual characteristic, and a central panel in the front belt region or the back belt region comprising a second visual characteristic.

### CROSS-REFERENCE TO RELATED APPLICATIONS

This application is a continuation of and claims priority under 35 U.S.C. § 120 to PCT Patent Application Serial No. PCT/CN2021/141087 filed on Dec. 24, 2021, the entire disclosure of which is hereby incorporated by reference.

### FIELD

This disclosure relates to disposable absorbent articles, such as diapers, having a more underwear-like article. The absorbent article may have improved communicative properties.

### BACKGROUND

Disposable absorbent articles which have a conforming, underwear-like looking are more desirable for moms and babies because it allows them to aspire to a time when the baby will be potty trained and no longer in diapers. Thus, an absorbent product that can deliver an underwear-like perception is more desirable than one that does not. There are, however, many obstacles to designing and executing an absorbent article that is underwear-like. One is that absorbent articles are a compilation of separate article components and as new disposable absorbent article technologies are developed, they may result in the need for incorporation of additional, separate, article components. Because of the manner in which absorbent article components are incorporated, even the viewable surfaces of the article may have seams or areas of overlap or connection. Thus, it is challenging to place graphics on these individual components and to line them up such that the multi-component construction appears to be an integrated structure wherein the areas of overlap are de-emphasized and process variations are masked.

Further, depending on the type of article construction, it is challenging to maintain the uniform appearance of graphics that may be printed or textured on more than one component. For example, a component with a particular graphic printed on it may eventually be incorporated into the article at a different depth than an adjacent component that also has a graphic printed on it. Depending on which component particular portions of a graphic are printed on for example, or, if not, how opaque the layers may be through which the various portions of the graphic may be viewed, can have an impact on an entire article graphic that is meant to look uniform and consistent in that the various portions may have measurable differences in appearance when printed on different components or at different depths relative to the outer surface.

Some disposable absorbent articles are configured in an open form (e.g., taped diapers) so they can be fastened around a wearer's torso and adjusted accordingly. Despite being an open form product, it is still desirable for the product to be perceived like underwear. Due to the structural difference in parts of an absorbent article, especially in belt areas, the absorbent article may be disadvantageous in providing an integral appearance.

It is thus desirable to provide absorbent articles that look like underwear, while, at the same time, incorporating multiple absorbent article components that offer the benefits and functionality of the latest technologies.

It is thus desirable to provide absorbent articles having a holistic perception belt design to convey underwear-like looking.

It is also desirable to provide absorbent articles which can visualize stretchability in a stretchable belt area which enables to communicate a better fit perception.

### SUMMARY

The present invention relates to an absorbent article continuous in a longitudinal direction, comprising: a front region, a back region, a crotch region disposed between the front and back regions; and a topsheet, a backsheet, and an absorbent core disposed between the topsheet and the backsheet, the backsheet being disposed to cover continuously the crotch region and at least part of the front region and back region in the longitudinal direction,

wherein the front region comprises a front belt region which comprises a first front belt substrate, a left side panel, a right side panel and a central panel between the left side panel and a right side panel, or the back region comprises a back belt region which comprises a first back belt substrate, a left side panel, a right side panel and a central panel between the left side panel and a right side panel,

wherein the first front belt substrate or the first back belt substrate overlaps the backsheet in at least part of the central panel,

wherein the left and right side panels in the front belt region or the back belt region comprise a first visual characteristic, and the central panel in the front belt region or the back belt region comprises a second visual characteristic.

For ease of discussion, the absorbent article will be discussed with reference to the numerals referred to in the Figures. The Figures and detailed description should however not be considered limiting the scope of the claims, unless explicitly indicated otherwise, and the invention disclosed herein is also used in a wide variety of absorbent article forms.

### BRIEF DESCRIPTION OF THE DRAWINGS

FIG. 1 shows a schematic perspective view of an exemplary absorbent article in a closed configuration as it would be when worn by a wearer.

FIG. 2 shows a plan view of a garment-facing side of an exemplary diaper according to the present invention with the diaper flattened out.

FIG. 3 shows a plan view of a wearer-facing side of an exemplary diaper according to the present invention with the diaper flattened out.

FIG. 4 is a lateral cross-section along 4-4 of the diaper of FIG. 3.

FIG. 5 is a lateral cross-section along 5-5 of the diaper of FIG. 3.

FIG. 6 is a lateral cross-section along 5-5 of another embodiment of the diaper of FIG. 3.

FIG. 7 is a lateral cross-section along 7-7 of the diaper of FIG. 3.

FIG. 8 is a lateral cross-section along 7-7 of another embodiment of the diaper of FIG. 3.

FIG. 9 is a plan view of a garment-facing side of an exemplary diaper according to the present invention with the diaper flattened out.

FIG. 10 is a partial view of the diaper of FIG. 9 showing a back region of the diaper.

FIG. 11 is a partial view of the diaper of FIG. 9 showing a front region of the diaper.

### DETAILED DESCRIPTION

As used herein, the following terms shall have the meaning specified thereafter:
“Absorbent article” refers to devices which absorb and contain body exudates and, more specifically, refers to devices which are placed against or in proximity to the body of the wearer to absorb and contain the various exudates discharged from the body. Exemplary absorbent articles include diapers, training pants, pull-on pant-type diapers, refastenable diapers, incontinence briefs and undergarments, diaper holders and liners, feminine hygiene garments such as panty liners, absorbent inserts, and the like.
‘Color’ or ‘colored’ as referred to herein includes any primary color except white, i.e., black, red, blue, violet, orange, yellow, green, and indigo as well as any declination thereof or mixture thereof.
“Disposed” refers to an element or a material being located in a particular place or position.
“Elastomeric material” or “elastic material” are used interchangeably and refer to a material that, upon application of a biasing force, can stretch to an elongated length of at least about 110% of its relaxed, original length (i.e. can stretch to 10 percent more than its original length), without rupture or breakage, as measured by EDANA method 20.2-89, and recover at least 40% of its elongation upon release of the applied force. For example, an elastic material that has an initial length of 100 mm can extend at least to 150 mm, and upon removal of the force retracts to a length of at least 130 mm (i.e., exhibiting a 40% recovery).
“Film” refers to a sheet-like material wherein the length and width of the material far exceed the thickness of the material. Typically, films have a thickness of about 0.5 mm or less.
“Graphic” refers to a colored visual presentation.
“Inboard” and “Outboard” refer respectively to the location of an element relative a centerline (longitudinal or lateral) of the absorbent article. Inboard refers to a location closer to the centerline and outboard refers to a location further from the centerline.
“Joined” refers to configurations whereby an element is directly secured to another element by affixing the element directly to the other element and to configurations whereby an element is indirectly secured to another element by affixing the element to intermediate member(s) which in turn are affixed to the other element.
“Longitudinal” refers to a direction running substantially perpendicular from a waist edge to an opposing waist edge of the article and generally parallel to the maximum linear dimension of the article. Directions within 45 degrees of the longitudinal direction are considered to be “longitudinal”
“Lateral” refers to a direction running from a longitudinal edge to an opposing longitudinal edge of the article and generally at a right angle to the longitudinal direction. Directions within 45 degrees of the lateral direction are considered to be “lateral.”
“Texture” is used to describe the individual's perception of the spatial variation of visible light due to surface structure of a portion of an object in two of a given material as a result of the material formation process. Textures can also be imparted to a material using techniques known to those skilled in the art including, for example, printing, embossing, bonding, aperturing and the like.
“Visual characteristic” refers to a visible, distinguishing or recognizable feature or attribute. Visual characteristics include graphic, color, texture, and the like.
“Visible” is intended to mean attribute of feature that is visually perceived by an individual user or consumer. Generally for a consumer or user, the attribute should be visible in the range of about 0.25 feet (0.075 meters) to about 3 feet (0.91 meters).
“Wearer-facing” and “garment-facing” refer respectively to the relative location of an element or a surface of an element or group of elements. “Wearer-facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable absorbent article).

### Absorbent Article

The absorbent articles of the present invention can be generally described with reference to a taped diaper 20 shown in Figures. The Figures are used herein as illustration of one way to carry out the invention and are not limiting the scope of the claims, unless specifically indicated to do so. In the following, the word “diaper” and “absorbent article” are used interchangeably.

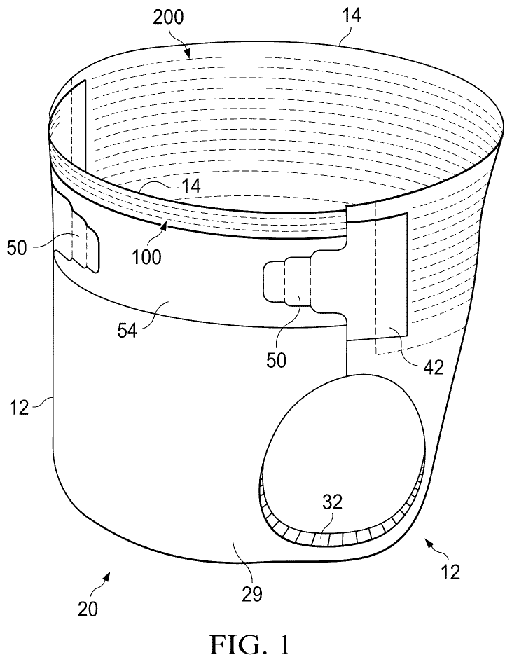

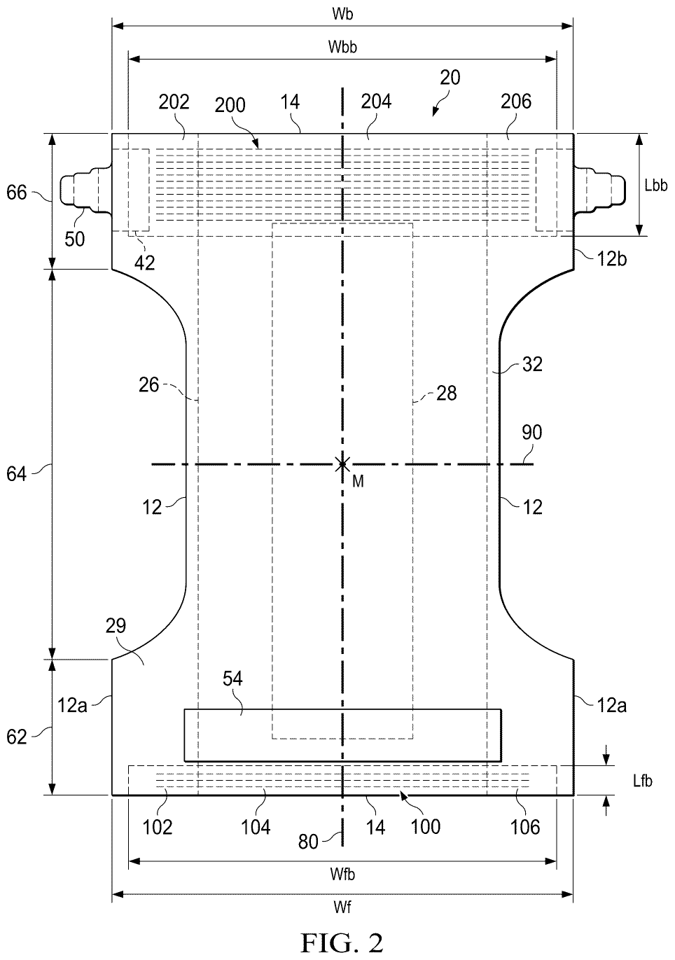

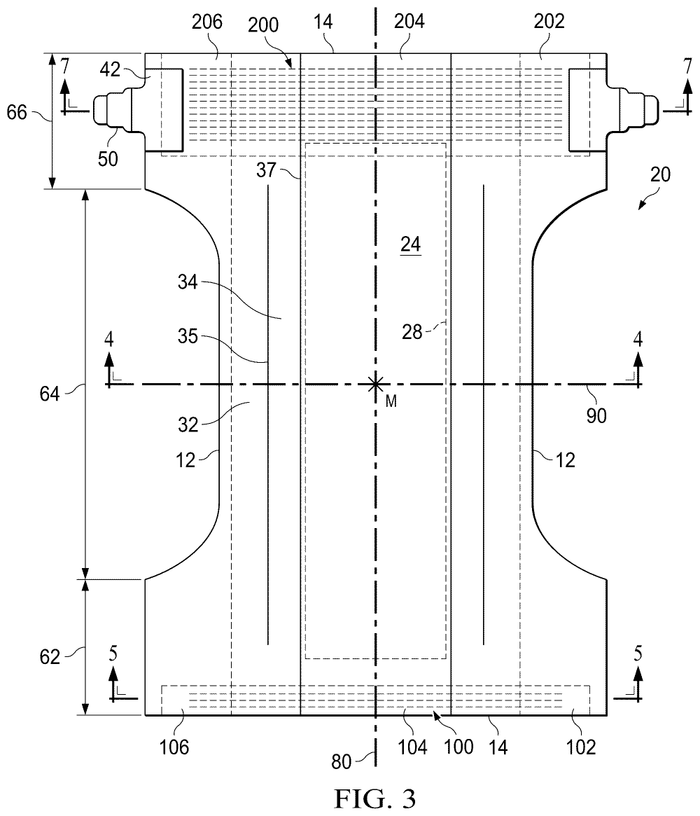

FIG. 1 is a perspective view of a diaper 20 in a closed configuration as it would be when worn by a wearer. FIG. 2 shows a top view of the garment-facing side of the diaper with the tapes open in a flat, uncontracted state (i.e., without elastic induced contraction), and the diaper stretched flat, and FIG. 3 shows the same diaper from the wearer-facing side. Referring to FIGS. 2 and 3, the absorbent article 20 includes a longitudinal centerline 80 and a lateral centerline 90. The absorbent article 20 is shown to have a front region 62, a back region 66 opposed to front region 62, and a crotch region 64 located between the front region 62 and the back region 66. Crotch region 64 is that portion of the absorbent article 20 which, when the absorbent article 20 is worn, is generally positioned between the legs of the wearer. The front and back regions 62 and 66 generally comprise those portions of the absorbent article 20 which, when worn, encircle the waist of the wearer. The front region 62 has a width W_{f} in the direction parallel to the lateral centerline 90. The back region 66 has a width W_{b} in the direction parallel to the lateral centerline 90.

At least one of the front region 62 and the back region 66 comprises a belt region (front belt region 100, and back belt region 200, respectively). That is, the front region 62 comprises a front belt region 100, and/or the back region 66 comprises a back belt region 200. The term “a belt region” can indicate either a front belt region 100 or a back belt region 200, or both the front belt region 100 and the back belt region 200 collectively.

The absorbent article 20 comprises a liquid permeable topsheet 24, a backsheet 26, and an absorbent core 28 between the topsheet 24 and the backsheet 26. The absorbent article 20 my further comprises an outer cover 29 disposed on a garment-facing side of the backsheet 26 to forming at least part of the garment-facing surface of the absorbent article. It should be recognized that other structures, elements, or substrates may be positioned between the core 28 and the topsheet 24 and/or backsheet 26.

The outer periphery of the absorbent article is defined by longitudinal edges 12 and waist edges 14. The longitudinal edges 12 may be subdivided into a front longitudinal edge 12a, which is the portion of the longitudinal edge 12 in the front region 62, and a rear longitudinal edge 12b, which is the portion of the longitudinal edge 12 in the back region 66. The absorbent article 20 has opposing longitudinal edges 12 that are oriented generally parallel to the longitudinal centerline 80. However, for better fit, longitudinal edges 12 may be curved or angled to produce, for example, an “hourglass” shape diaper when viewed in a plan view. The absorbent article 20 has opposing waist edges 14 that are oriented generally parallel to the lateral centerline 90.

The exemplary taped diaper shown in Figures has a unibody construction, with shaped longitudinal edges 12 formed by the combination of the secondary topsheet layer 24b on the wearer-facing side and the outer cover 29 on the garment-facing side. However the invention is applicable to any kind of absorbent article construction, including taped diapers having a chassis formed by a backsheet and topsheet having generally straight longitudinal edges 12, the diaper then comprising discrete front cars and back cars that are attached to the chassis, as is also known in the art.

### Topsheet

The topsheet 24 is the part of the absorbent article 20 that is in contact with the wearer's skin. The topsheet 24 may be joined to portions of the backsheet 26, the absorbent core 28, the barrier leg cuffs 32, 34, and/or any other layers as is known to those of ordinary skill in the art. The topsheet 24 should be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments, or a combination of natural and synthetic fibers.

The topsheet may be apertured, may have any suitable three-dimensional features, and/or may have a plurality of embossments (e.g., a bonding pattern).

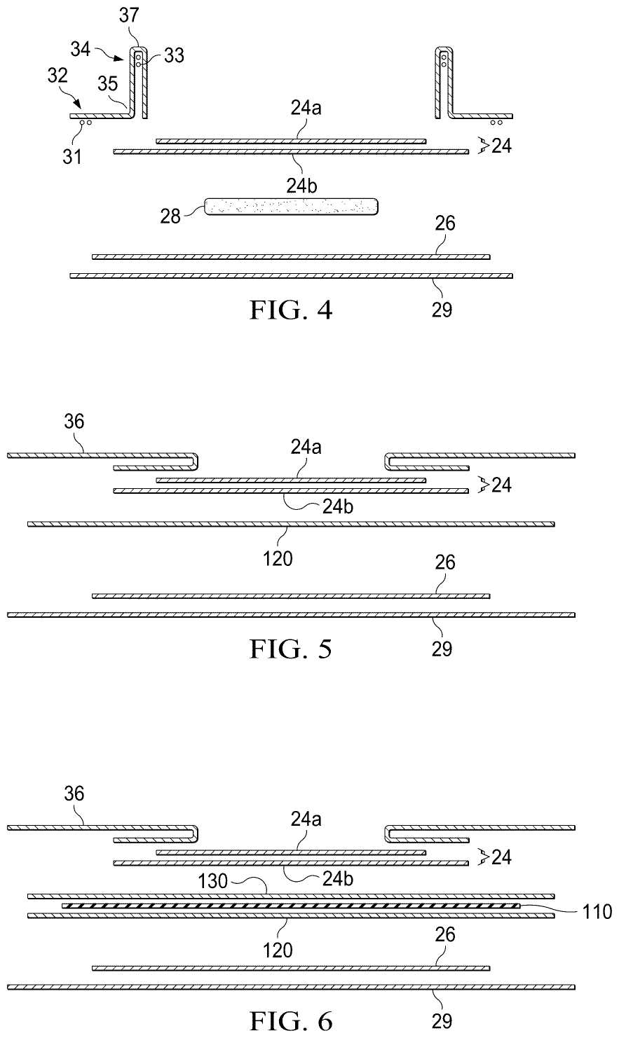

The topsheet may be a single layer, or may also have one or more layers, for example as represented in the FIGS. 4-8 as a dual layer laminate construction comprising a primary topsheet layer 24a disposed on top of a secondary topsheet layer 24b. The primary topsheet layer 24a alone, or the primary topsheet layer 24a and the secondary topsheet layer 24b together may have a three-dimensional features.

The topsheet typically consists of or comprises at least a nonwoven layer. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers.

### Absorbent Core

As used herein, the term “absorbent core” refers to a component disposed in the article for absorbing and containing liquid such as urine received by the absorbent article. The absorbent core thus typically has a high absorbent capacity. The absorbent core comprises an absorbent material, that may be enclosed within or sandwiched between a core wrap (not represented for simplicity). An absorbent core does not include the acquisition and/or distribution system or any other components of the article which are not either integral part of the core wrap or placed within the core wrap or core bag.

The core wrap may be a single material that is folded and attached to itself, or it may comprise a separate top layer and bottom layer that may be bonded or otherwise joined together. The absorbent material may comprise superabsorbent particles which are optionally mixed with cellulose fibers.

The absorbent material may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. The absorbent material may also be free of cellulose fibers, as is known in so-called airfelt-free cores, where the absorbent material consists, or consists essentially, of SAP. The absorbent material may also be a high internal phase emulsion foam.

“Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from 20 g/g to 40 g/g. “Superabsorbent polymer particles”, as used herein, refers to a superabsorbent polymer material which is in particulate form so as to be flowable in the dry state.

The absorbent core 28 may comprise a high amount of SAP enclosed within the core wrap. The SAP content may represent 70%-100% or at least 70%, 75%, 80%, 85%, 90%, 95%, 99%, or 100%, by weight of the absorbent material, contained in the core wrap. The core wrap is not considered as absorbent material for the purpose of assessing the percentage of SAP in the absorbent core. The core may also contain airfelt or cellulosic fibers with or without SAP.

Various absorbent core designs comprising high amounts of SAP have been proposed in references. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. These glues are not represented in the Figures for simplicity. Other core constructions comprising a high loft nonwoven substrate such as a carded nonwoven layer, having a porous structure into which SAP particles have been deposited, may also be used in present disclosure.

The basis weight (amount deposited per unit of surface) of the absorbent material may also be varied to create a profiled distribution of absorbent material, in particular in the longitudinal direction to provide more absorbency towards the center and the middle of the core, but also in the lateral direction, or both directions of the core.

The absorbent material layer may include one or more channel-forming area(s). Channel-forming areas are substantially free of absorbent material, in particular areas completely free of absorbent material (accidental minute amount of absorbent material due to involuntary contamination of the channels due to the high speed of the making process being disregarded). The channel-forming area(s) when present may comprise a channel bond between the top side of the core wrap and the bottom side of the core wrap. This bond provides for structural integrity of the channels in dry and wet state. Any known bonding techniques known in the art may be used to provide for this bond, but in particular a hotmelt adhesive bond may be used for the channel bond(s). An adhesive may be for example applied in the areas of the channels on the inner side of the top side and/or the inner side of the bottom side of the core wrap, typically by slot glue application or any other means, followed by the application of pressure in the areas of the channels to provide a good adhesive bonding in these areas. Exemplary patent disclosures of such adhesive bonding processes can be found for an airfelt or airfelt-free absorbent cores in WO2012/170,798A1 (Jackels et al.), EP2,905,000 (Jackels et al.) and EP2,905,001 (Armstrong-Ostle et al.).

### Backsheet

The backsheet 26 is generally positioned such that it may be at least a portion of the garment-facing surface of the absorbent article 20. Backsheet 26 may be designed to prevent the exudates absorbed by and contained within the absorbent article 20 from soiling articles that may contact the absorbent article 20, such as bed sheets and undergarments. The backsheet may be joined to portions of the topsheet 24, the absorbent core 28, the optional outer cover material 29, and/or any other layers of the absorbent article by any attachment methods known to those of skill in the art. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet 26 materials may include breathable materials that permit vapors to escape from the absorbent article 20 while still preventing exudates from passing through the backsheet 26. Exemplary breathable materials may include materials such as woven webs, nonwoven webs, composite materials such as film-coated nonwoven webs, and microporous films. Other suitable materials and/or manufacturing techniques may be used to provide a suitable backsheet 26 including, but not limited to, surface treatments, particular film selections and processing, particular filament selections and processing, etc.

### Outer Cover

The absorbent article 20 may comprise an outer cover 29 constituting at least part of a garment-facing surface of the absorbent article 20. An outer cover layer 29, when the absorbent article 20 may further comprise it, is disposed on the garment-facing side of the backsheet 26. When the absorbent article is formed by a unibody chassis as represented in the Figures, the outer cover 29 typically extends laterally longitudinal edges of the backsheet 26 so that it can form with the topsheet (or one layer of the topsheet for a laminate) the external edges of the absorbent article, in particular the longitudinal edges of the absorbent article.

The outer cover 29 may comprise one or more nonwoven materials joined to the backsheet 26. The outer cover 29 forms at least a portion of the garment-facing surface of the absorbent article 20 and effectively “covers” the backsheet 26 so that the backsheet is not present on the garment-facing surface. The outer cover 29 may comprise a bond pattern, apertures, and/or three-dimensional features.

### Inner Cuffs and Outer Cuffs

The absorbent article 20 may comprise a pair of barrier leg cuffs 34 (also called inner cuffs) and a pair of gasketing elastic cuffs 32 (also called outer cuffs), as is known in the art and as briefly explained herein. The barrier leg cuffs 34 are positioned laterally inboard of gasketing cuffs 32 and can extend vertically away from the chassis of the article, as shown in FIG. 4. The barrier leg cuffs 34 are thus delimited by a proximal edge 35 joined directly or indirectly to the topsheet and/or the backsheet and a free terminal edge 37, which is intended to contact and form a seal with the wearer's skin. The barrier leg cuffs 34 typically extend partially between the front and back waist edges 14 on opposite sides of the central longitudinal centerline 80 and are at least present at the level of the M point. The barrier leg cuffs 34 preferably comprise one or more elastics strands 33 near or at the free terminal edge 37. These elastics 33 cause the barrier leg cuffs 34 to form a seal around the legs and torso of a wearer.

The gasketing elastic cuffs 32 are elasticized and preferably comprise one or more elastic strands 31 extending at least partially between the front and back waist edges 14 of the article, and are at least present at the level of the point M. The gasketing cuffs elastics 31 essentially cause portions of the absorbent article proximate to the longitudinal edges 12 to form a seal around the legs of the wearer.

Both outer and inner cuffs 32, 34 may be formed by a piece of material 36, typically a nonwoven such as a SMS, which is partially bonded to the absorbent article 20 along the proximate edge 35 of the cuff so that the inner cuffs 34 can extend upwards from a wearer-facing surface of the absorbent article 20 and provide improved containment of body exudates approximately at the junction of the torso and legs of the wearer.

### Landing Zone

Referring to FIGS. 1-3, the absorbent article 20 in the form of a taped diaper may have a discrete material forming a landing zone 54 on its garment-facing side, typically disposed proximate the front edge 14 of the article 20. The landing zone 54 is configured to receive the fasteners 50 and may comprise, for example, a plurality of loops configured to be engaged with, a plurality of hooks on the fasteners, or vice versa.

The landing zone 54 may comprise a nonwoven material that is attached to a portion of the outer cover 29 adjacent to the front waist edge 14 of the diaper 20. The outer cover 29 may also serve directly as a landing zone without the need of a discrete landing zone 54, if the outer cover has sufficient loft or other required properties to sufficient interact with the fasteners 50.

### Ears

The absorbent article 20 may include back car and/or front cars (collectively, “ears”). The back cars 42 may be extensible, inextensible, elastic, or inelastic. In certain embodiments the back car 42 may be formed of a stretch laminate such as a nonwoven/elastomeric material laminate or a nonwoven/elastomeric material/nonwoven laminate. The cars may be discrete or integral. A discrete car is formed as separate element which is joined to a main body of the absorbent article. An integral car is a portion of the main body that projects laterally outward from the longitudinal edge 12. The integral ear may be formed by cutting the chassis form to include the shape of the car projection. When the absorbent article 20 further comprises back cars, the back cars may comprise fastening members 52. also include a fastening system 50. When fastened, the fastening system 50 interconnects the front region 62 and the back region 66 resulting in a waist circumference that may encircle the wearer during wear of the absorbent article 20.

### Belt Regions

At least one of the front region 62 and the back region 66 comprises a belt region 100, 200 comprising a first belt substrate e120, 220. That is, the front region 62 comprises a front belt region 100 comprising a first front belt substrate 120, and/or the back region 66 comprises a back belt region 200 comprising a first back belt substrate 220. The term “a first belt substrate” can indicate either a first front belt substrate 120 or a first back belt substate 220, or both the first front belt substrate 120 and the first back belt substate 220 collectively.

Referring to FIG. 5, when the front region 62 comprises a front belt region 100, the front belt region 100 comprises a first front belt substrate 120 which defines the front belt region 100. The front belt region 100 may extend to crotch region 64, so that the front belt region 100 is disposed to cover continuously at least part of the front region 62 and part of the crotch region 64.

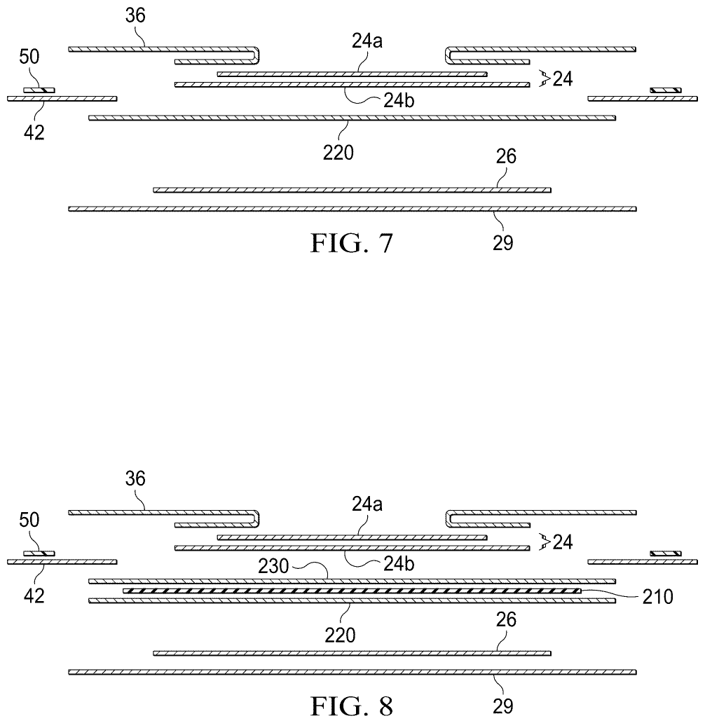

Referring to FIG. 7, when the back region 66 comprises a back belt region 200, the back belt region 200 comprises a first back belt substrate 220 which defines the back belt region 200. The back belt region 200 may extend to crotch region 64, so that the back belt region 200 is disposed to cover continuously at least part of the back region 66 and part of the crotch region 64.

The first front belt substrate 120 and/or the first back belt substrate 220 may comprise spunbond nonwoven.

The belt region 100, 200 may be directly adjacent to (i.e. less than 10 mm, or less than 0.5 mm away from) the front or back edge 14 of the absorbent article. The belt region 100, 200 has a longitudinal edge substantially aligned with the front edge 14 or the back edge 14 in a z-direction (a product thickness direction).

The first front belt substrate 120 and the first back belt substrate 220 may be the same material or different materials.

The front and back belt regions 100 and 200 in a relaxed product have lengths L_{fb} and L_{bb} respectively in the direction parallel to the longitudinal centerline 80 of the absorbent article 20. The front and back belt regions 100 and 200 in a relaxed product have widths W_{fb} and W_{bb} respectively in the direction parallel to the lateral centerline 90 of the absorbent article 20. The front belt region 100 or the back belt region 200 in a relaxed product has a width in the direction parallel to the lateral centerline 90 of the absorbent article longer than a length of backsheet 26 in the direction parallel to the lateral centerline 90 of the absorbent article.

Referring to FIGS. 2 and 3, and 9-11, the front belt region 100 comprises a left side panel 102, a right side panel 106, and a central panel 104 between the left side panel 101 and a right side panel 106, and the back belt region 200 comprises a left side panel 202, a right side panel 206 and a central panel 204 between the left side panel 202 and a right side panel 206.

The first belt substrate 120, 220 overlaps backsheet 26 in at least part of the central panel 104, 204. The central panel 104, 204 has a width in the direction parallel to the lateral centerline 90 of the absorbent article defined by a length of backsheet 26 in the direction parallel to the lateral centerline of the absorbent article.

The width W_{fb} of front belt region 100 may be at least about 80%, at least about 85%, or at least about 90% of the width W_{f} of the front region 62 in the direction parallel to the lateral centerline of the absorbent article. If the width W_{fb} of front belt region 100 is too short, the absorbent article may not provide a desirable wholistic appearance along the belt region.

The length L_{fb} of the front belt region 100 may be greater than about 10 mm, greater than about 15 mm or greater than about 20 mm. The length L_{fb} of front belt region 100 may be no greater than about 160 mm, no greater than about 120 mm, or no greater than about 30 mm. If the front belt region length L_{fb} is too short, the limited length of the front belt region makes it difficult to place visual characteristic and/or enough number of clastic strands.

When the absorbent article 20 optionally comprises a discrete landing zone 54 as shown in the Figures, the landing zone 54 may be advantageously disposed underneath the front belt region 100. In other words, the front belt region 100 may be advantageously disposed between the landing zone 54 and the front edge 14 of the absorbent article 20. The belt region 100 may overlap at least partially the landing zone 54.

The width W_{bb} of back belt region 200 may be at least about 80%, at least about 85%, at least about 90% of the width of W_{b} of the back region 66 in the direction parallel to the lateral centerline of the absorbent article. If the width W_{bb} of back belt region 200 is too short, the absorbent article may not provide a desirable wholistic appearance along the belt region/

The length L_{bb} of back belt region 200 may be greater than about 20 mm, greater than about 40 mm, or greater than about 60 mm. The length L_{bb} of back belt region 200 may be no greater than about 160 mm, no greater than about 120 mm, or no greater than about 90 mm. If the length L_{bb} of back belt region 200 is too short, the back belt region may cause skin marks especially when the back belt region comprises an elastic material.

The front belt region 100 and/or the back belt region 200 may further comprise an elastic material. Referring to FIG. 6, the front belt region 100 may further comprise a second front belt substrate 130 in addition to the first front belt substrate 120, and at least one clastic strand 110 disposed between the first front belt substrate 120 and the second front belt substrate 130. Referring to FIG. 8, the back belt region 200 may further comprise a second back belt substrate 230 in addition to the first back belt substrate 220, and at least one clastic strand 210 disposed between the first back belt substrate 220 and the second back belt substrate 230.

Elastic strands can be provided to the belt region 100, 200 by various methods and apparatus well known to those skilled in art. For example, clastic strands 110, 210 are provided in the belt region 100, 200 by advancing the first belt substrate 120, 220 and the second belt substrates 130, 230 and a plurality of clastic strands 110, 210 in the machine direction, stretching the clastic strands 110, 210 in the machine direction, and adhering certain portions of the three components one another. The adhesion may be provided by holt-melt adhesive method, heat bonding, ultrasonic bonding, or any other method known in the art. By such steps and after the tension stretching the elastic strands are eventually removed, the elastic strands return to their relaxed state to create gathers with the first belt substrate 120, 220 and the second belt substrate 130 and 230. The clastic strand 110, 210 may be positioned in any interval between each other, in any length in a longitudinal direction to meet purposes of provision of graphics as well as desirable elasticity to the article.

When both of the front and back regions of the absorbent article of the present invention comprise elastomeric materials, it may be more desirable that the front region has a lower elastic force that the back region.

The front belt region 100 comprises a first visual characteristic disposed in the left and right panels 102, 106 and a second visual characteristic disposed the central panel 104. The back belt region 200 comprises a first visual characteristic disposed in the left and right panels 202 and 206, and a second visual characteristic disposed the central panel 204. The visual characteristics are not represented in FIGS. 1-3, and 5-8 for simplicity.

### Visual Characteristics

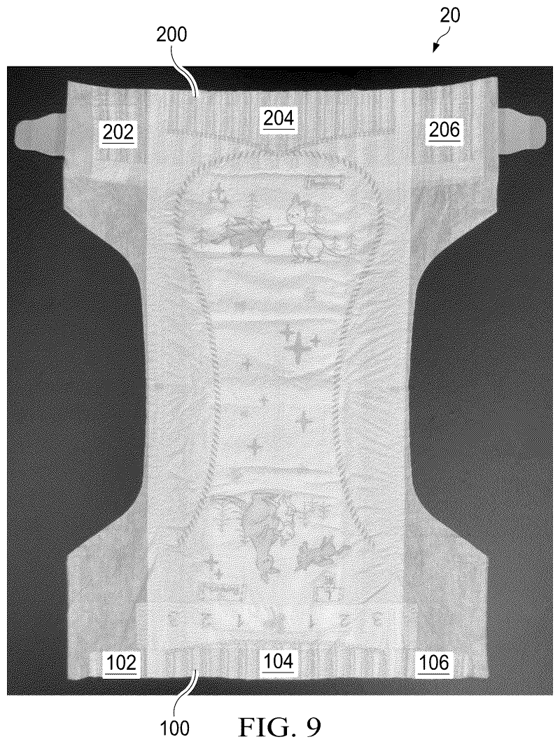

Referring to FIGS. 9-11, when the absorbent article 20 comprises a front belt region 100 which comprises a left side panel 102, a right side panels 106, and a central panels 104 disposed between the left and right side panels 102, 106, the left and right side panels 102, 106 comprise a first visual characteristic, and the central panel 104 comprises a second visual characteristic. Still referring to FIGS. 9-11, the absorbent article 20 comprises a back belt region 200 which comprises a left side panel 202, a right side panels 206, and a central panels 204 disposed between the left and right side panels 202, 206, the left and right side panels 202, 206 comprise a first visual characteristic, and the central panel 204 comprises a second visual characteristic.

The term “a visual characteristic” can indicate either a first visual characteristic or a second visual characteristic, or both the first visual characteristic and the second visual characteristic collectively.

The visual characteristic may be a feature or attribute selected from the group consisting of graphic, color, texture, and combinations thereof. In some embodiments, the first visual characteristic and the second visual characteristic have a common element such as graphic, color, texture, and the like. In such embodiments, the first visual characteristic and the second visual characteristic may be coordinated. The first visual characteristic and the second visual characteristic are considered when they have substantially the same graphic or shape even though they are of different size, notwithstanding that additional shape elements or in different color are present; or when they have substantially the same color even though they are of different graphic or shape, notwithstanding that additional graphic elements are present.

A first visual characteristic in the front belt region 100 and a first visual characteristic in the back belt region 200 may be coordinated. A first visual characteristic in the front belt region and a first visual characteristic in the back belt region may be distinctively different from each other.

The visual characteristic may be continuous or discontinuous. In some embodiments, the visual characteristic are discontinuous.

In one embodiment, the first visual characteristic is disposed on the first belt substrate, and the second visual characteristic is disposed on the backsheet 26.

In one embodiment, the first visual characteristic is a first color or a first graphic, and the second visual characteristic is a second color or a second graphic. The first color and/or graphic may be disposed on an outer surface of the first belt substrate. The first color and/or the first graphic may be disposed on an outer (garment-facing) surface of the first belt substrate, and the second color and/or graphic may be disposed on an outer (garment-facing) surface of backsheet 26. When the first visual characteristic comprises color or graphic, and the second visual characteristic comprises color or graphic, the first visual characteristic has a first lightness no greater than about 73, or no greater than about 70, or no greater than 68 as measured by Lightness Measurement disclosed herein, and the second visual characteristic has a second lightness no greater than about 73, or no greater than about 70, or no greater than 68 as measured by Lightness Measurement disclosed herein. The difference between the first lightness and the second lightness may be no greater than about 6, no greater than about 5, or no greater than about 4. Without being bound by theory, when the first or the second visual characteristic has a lightness no greater than about 73, and the difference between the first lightness and the second lightness is no greater than about 6, an entire article graphic in the belt region is meant to look uniform, and can create wholistic continuous appearance.

Visual characteristics disclosed herein can be provided by various materials, methods and apparatus well known to those skilled in art.

Any material applicable to a component consisting of a belt region and/or a backsheet of the absorbent article and/or creating a visual characteristic can be used. In some embodiments, the materials may be in a liquid form when applied to a first belt substrate or a backsheet and become solid after applied to, such as adhesive and printing ink. In other embodiments, the materials may be in a solid form when applied to a first belt substrate or a backsheet and remains a solid once it is applied, such as elastic strands, fibers and web.

The visual characteristics can be provided by printing inks. The first visual characteristic may be created by printing a color or graphic on an inner side and/or outer side of the first belt substrate 120, 220. The second visual characteristic may be disposed by printing a color or graphic on an inner side and/or outer side of the backsheet 26. In one embodiment, the first visual characteristic is printed on an outer side of a first belt substrate (first front belt substrate 120 and/or a first back belt substrate 220), and the second visual characteristic is printed on an outer side of the backsheet 26. In another embodiment, the second visual characteristic is printed on inner side of an outer cover 29 when the absorbent article further comprises an outer cover.

The visual characteristics when provided by printing inks, can be printed by various methods and apparatus well known to those skilled in the art such as lithographic, screen printing, flexographic, gravure ink jet printing techniques or a method, and virtually any graphic in any color or color combination can be rendered on the sheet.

When the front belt region 100 comprises a first front belt substrate 120, a second front belt substate 130, and a plurality of elastic strands 110 sandwiched between the first and second front bet substates 120, 130, or when the back belt region 200 comprises a first back belt substrate 220, a back second belt substate 230, and a plurality of elastic strands 210 sandwiched between the back first and second substates 220, 230, the first and second substrates and/or the elastic strands can be adhered with each other by adhesive.

Visual characteristics, for example the first visual characteristic, may be provided by one or more colored clastic strands of the plurality of elastic strands 110 or 210 disposed between the first belt substrate 120 or 230 and the second belt substrate 120 or 220 are colored elastic strands.

Visual characteristics may be provided by colored adhesive. In the front and back belt regions, the first and second belt substrates and/or the elastic strands can be joined to each other by colored adhesive. The backsheet can be joined to a first belt substrate and/or an outer cover by colored adhesive. The adhesive may be in the form of meltblown adhesive, spray coating resins, or web forming resins, and disposed in the inner side of either the outer or inner sheet. For example, hot melt adhesives are applied to an advancing web in pre-determined pattern by slot die coating, direct gravure, offset gravure or reverse gravure roll coating processes that are extensively described in the art. Adhesive may be applied in various patterns, on the first or the second belt substrates, the backsheet, and/or the outer cover. A shape of a pattern, a width and length of each pattern or an interval between two adjacent patterns can be determined to meet purposes of provision of graphics as well as binding the first and the second belt substrates, or boding the backsheet and the first belt substrate and/or the outer cover.

The first visual characteristic may be disposed along a width direction of the belt region 100, 200.

The first visual characteristic may be disposed directly adjacent to (i.e. less than 10 mm, or less than 5 mm away from) an outboard side edge of the left side panel or an outboard side edge of the right side panel.

With a first visual characteristic and a second visual characteristic, an absorbent article of the present invention may provide a holist belt perception along the waist edge which is preferable for a better underwear-look perception. In one embodiment, when the belt region comprises an elastic material, with a first visual characteristic and a second visual characteristic, an absorbent article of the present invention may provide a holist waist stretch perception which is preferable for a better underwear-look perception, and a better fit perception as a first visual characteristic may visualize a belt stretch area comprising the elastic material in the left and right side panels.

A width of an area in the direction parallel to the lateral centerline 90 of the absorbent article where the first visual characteristic or the second visual characteristic is disposed, and a length of the area in the direction parallel to the longitudinal centerline 80 of the absorbent article where the first visual characteristic or the second visual characteristic is disposed can be determined depending on various product design purposes.

### Bio-sourced Material

Any components of the absorbent article of the present invention can at least partially be comprised of bio-sourced content as described in US2007/0219521A1, US2011/0139658A1, US2011/0139657A1, US2011/0152812A1, US2011/0139662A1, and US2011/0139659A1. These components include, but are not limited to, topsheet nonwovens, backsheet films, backsheet nonwovens, barrier leg cuff nonwovens, superabsorbent material, upper and lower core wrap layer, adhesives, fastener hooks, and fastener landing zone nonwovens and film based.

The disposable absorbent article component may comprise a bio-based content value from about 10% to about 100% using ASTM D6866-10, method B, in another embodiment, from about 25% to about 75%, and in yet another embodiment, from about 50% to about 60% using ASTM D6866-10, method B.

In order to apply the methodology of ASTM D6866-10 to determine the bio-based content of any disposable absorbent article component, a representative sample of the disposable absorbent article component must be obtained for testing. Thereto, the disposable absorbent article component may be ground into particulates less than about 20 mesh using known grinding methods (e.g., Wiley® mill), and a representative sample of suitable mass taken from the randomly mixed particles.

### TEST METHODS

### 1. Lightness Measurement

This method measures the lightness of a visually discernable area in the front belt region in the front region or the back belt region in the back region from the garment-facing surface of the wearable article 20. It is based on analysis of a calibrated digital image of a sample surface using an image analysis program (a suitable program is ImageJ v. 1.53, National Institute of

Health, USA, or equivalent).

### Sample Preparation

Lay a sample absorbent article flat on a non-reflective white flat background plate. To flatten the absorbent article, stretch out the absorbent article using a hook plate to ensure no significant wrinkles or unevenness in the sample. Care should be taken to avoid folds, wrinkles, or other conditions which may affect image acquisition and analysis.

### Image Acquisition

An image of the target area is taken using a lab-built image acquisition system. The key components include a DSLR camera (Canon EOS 6D Mark 2, or equivalent) with a lens (EF 24-105 mm f/4L IS 2 USM lens or equivalent), a non-reflective white background plate, and a dome light (JM Vis Tec DLP-600×600-WHI, or equivalent). The dome light is placed 800 mm away from the absorbent article vertically. The sample is laid horizontally flat at the center of the white background plate, directly beneath and centered within the camera's field of view mounted 835 mm above. Suitable settings might be a focal length of the camera set to 70 mm, ISO: 100, F stop: 8.0, Shutter speed: 1/60 sec. Using a standard 18% gray card (e.g., Munsell 18% Reflectance (Gray) Neutral Patch/Kodak Gray Card R-27, available from X-Rite; Grand Rapids, MI, or equivalent), the camera's white balance is a custom set for the lighting conditions. An image is properly focused, captured, and saved as a 24-bit RGB TIF file. The resulting image must be at a resolution of at least 18 pixels/mm.

### Measurement of Lightness

To measure the lightness of a target area, begin by identifying the boundaries of a distinct target area. The boundary of a target area is recognized by visual discernment of differences compared to other area(s) in the sample. For example, an area boundary can be identified based on visually distinguishing in graphic or color over other area in the sample. Using the image analysis software, manually select five regions of interest (ROI). In case the ROI are selected in a target area which contains a visual characteristic, ensure the ROI to represent the visual characteristic. Lightness (L* value) of each of the ROI is measured using the CIE L*a*b* color system (CIELAB). The L* values of the ROI are measured, and their average is calculated and recorded as L* value of the target area.

### EXAMPLE

A taped diaper 20 of FIG. 9 was prepared with the following dimensions. The diaper 20 had configurations as generally illustrated in the FIGS. 2-4, 6 and 8, and comprised a first visual characteristic printed on the outer surface (garment-facing surface) of the first front belt substrate 120 and the first back belt substrate 220, and a second visual characteristic printed on the outer surface (garment-facing surface) of backsheet 26.

| | Dimensions in mm |
|---|---|
| Diaper 20 length | 480 |
| Core 28 length | 375 |
| Diaper 20 width | 335 |
| Front region width W_{f}, Back region width W_{b} | 335 |
| Front region length, Back region length | 104 |
| First front belt substate 120 width W_{fb}, | 310 |
| First back belt substrate 220 width W_{bb} | |
| First front belt substrate 120 length L_{fb} | 22 |
| First back belt substrate 220 length L_{bb} | 73 |
| Landing zone 54 width | 230 |
| Backsheet 26 width | 210 |
| 1st topsheet layer 24a width | 110 |
| 2nd topsheet layer 24b width | 142 |

The back belt region 200 comprised 11 elastane elastic strands each having a dtex of 620 and the front belt region 100 comprised 3 elastane elastic strands each having a dtex of 470.

With the first visual characteristic in side panels 102 and 106 in the back belt region 200, and in side panels 202 and 206 in the back belt region 200; and the second visual characteristic in the central panels 104, 204, the diaper 20 provides a holistic perception belt design and convey underwear-like looking.

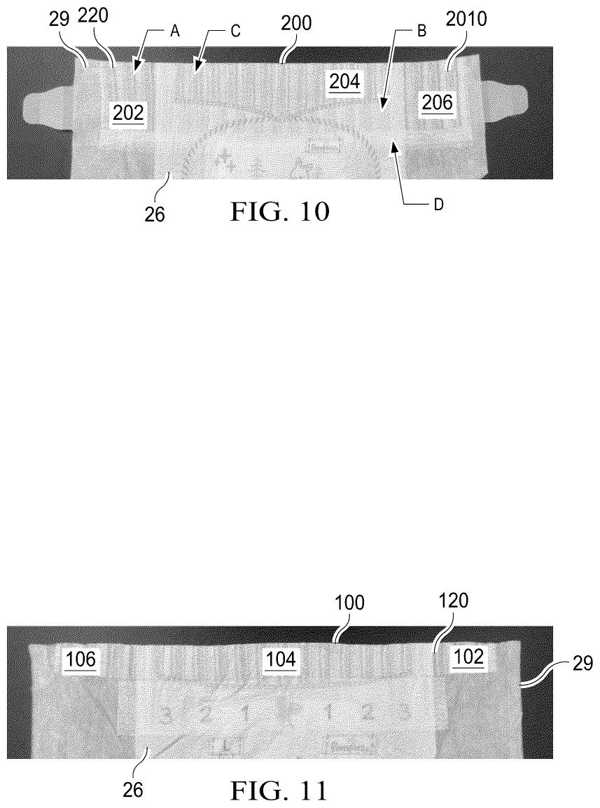

Referring to FIG. 10, lightness in each of four target areas A, B, C and D illustrated below in the back belt region 200 was measured according to the Lightness Measurement disclosed herein.

Area A: Area of first visual characteristic in the back left and right side panels 202, 206

Area B: Area having a visual characteristic printed on the first back belt substrate 220, and having no visual characteristic printed on the backsheet 26

Area C: Area of second visual characteristic in the back central panel 204

Area D: Area in the back central panel 204 having no visual characteristic either on the first back belt substrate 220, or on the backsheet

Results of Lightness are provided in Table 1 below.

TABLE 1

| Area | A | B | C | D |
|---|---|---|---|---|
| Lightness (L*) | 65.8 | 75.6 | 68.6 | 75.6 |

Area A having the first visual characteristic in side panels 202 and 206, and Area C having the second visual characteristic have a lightness significantly lower than Area B and Area D. The lightness difference between Area A and Area B is 2.8.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article continuous in a logitudinal direction comprising: a garment-facing surface, a front region, a back region, a crotch region disposed between the front and back regions; and a topsheet, a backsheet, and an absorbent core disposed between the topsheet and the backsheet, the backsheet being disposed to cover continuously the crotch region and at least part of the front region and back region in the longitudinal direction,
wherein the front region comprises a front belt region which comprises a first front belt substrate, a left side panel, a right side panel and a central panel between the left side panel and a right side panel; or the back region comprises a back belt region which comprises a first back belt substrate, a left side panel, a right side panel and a central panel between the left side panel and a right side panel,
wherein the first front belt substrate or the first back belt substrate overlaps the backsheet in at least part of the central panel,
wherein the left and right side panels in the front belt region or the back belt region comprise a first visual characteristic, and the central panel in the front belt region or the back belt region comprises a second visual characteristic.

2. The absorbent article of claim 1, wherein the back region comprises a back belt region comprising a first back belt substrate, a left side panel, a right side panel and a central panel between the left side panel and a right side panel, the first back belt substrate overlapping the backsheet in at least part of the central panel, and wherein the left and right side panels in the back belt region comprise a first visual characteristic, and the central panel in the back belt region comprises a second visual characteristic.

3. The absorbent article of claim 2, wherein the first visual characteristic is disposed on the first front belt substrate, and the second visual characteristic is disposed on the backsheet, or the first visual characteristic is disposed on the first back belt substrate, and the second visual characteristic is disposed on the backsheet.

4. The absorbent article of claim 2, wherein the first visual characteristic is a feature selected from the group consisting of color, graphic, texture, and combinations thereof; and the second visual characteristic is a feature selected from the group consisting of color, graphic, texture, and combinations thereof.

5. The absorbent article of claim 2, wherein the first visual characteristic and the second visual characteristic have a common element, the common element being an element selected from the group consisting of color, graphic, texture, and combinations thereof.

6. The absorbent article of claim 2, wherein the first visual characteristic and the second visual characteristic are coordinated.

7. The absorbent article of claim 1, wherein the number of layers between the first visual characteristic and the garment-facing surface is the same as the number of layers between the second visual characteristic and the garment-facing surface.

8. The absorbent article of claim 1, wherein the first visual characteristic is a first graphic or first color, and the second visual characteristic is a second graphic or second color.

9. The absorbent article of claim 8, wherein the first visual characteristic has a first lightness as measured by Lightness Measurement and the second visual characteristic has a second lightness as measured by Lightness Measurement, wherein the first lightness and the second lightness are no greater than about 73.

10. The absorbent article of claim 9, wherein the difference between the first lightness and the second lightness is no greater than about 6.

11. The absorbent article of the claim 1, wherein the first front belt substrate or the first back belt substrate comprises a spunbond nonwoven.

12. The absorbent article of claim 1, wherein the front region comprises a belt region, a first front belt substrate, a left side panel, a right side panel and a central panel between the left side panel and a right side panel, the first front belt substrate overlapping the backsheet in at least part of the central panel; and wherein the left and right side panels in the front belt region comprise a first visual characteristic, and the central panel in the front belt region comprises a second visual characteristic.

13. The absorbent article of claim 12, wherein the first visual characteristic in the front belt region and the first visual characteristic in the back belt region have a common element, the common element being an element selected from the group consisting of color, graphic, texture, and combinations thereof.

14. The absorbent article of claim 13, wherein the first visual characteristic in the front belt region and the first visual characteristic in the back belt region are coordinated.

15. The absorbent article of claim 1, wherein the back belt region further comprises an elastic material.

16. The absorbent article of claim 1, wherein the back belt region further comprises a second back belt so that the elastic material is disposed between the first back substrate and the second back belt substrate.

17. The absorbent article of claim 1, wherein the front belt region further comprises an elastic material.

18. The absorbent article of claim 1, wherein the absorbent article is an open form absorbent article.

19. The absorbent article of claim 1, wherein the absorbent article further comprises an outer cover layer disposed on a garment-facing side of the backsheet.
